(19) United States
(12) Patent Application Publication
(10) Pub. No.: US 2009/0264895 A1
(43) Pub. Date: Oct. 22, 2009

(54) SYSTEMS AND METHODS FOR IMPLANTING A BONE FASTENER AND DELIVERING A BONE FILLING MATERIAL

(75) Inventors: Allison C. GASPERUT, Memphis, TN (US); William A. Rezach, Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedic, Inc., Warsaw, IN (US)

(21) Appl. No.: 12/107,440

(22) Filed: Apr. 22, 2008

(51) Int. Cl. A61B 17/58; B25C 7/00

(52) U.S. Cl. 606/104; 227/141

(57) ABSTRACT

A driver for fastening a bone fastener to a bone, the driver comprises an elongated outer member including a first bore extending therethrough along a longitudinal axis and a coupling element that is releasably coupled with the bone fastener. The driver further comprises an elongated material conduit extending at least partially within the first bore. The material conduit including a second bore extending therethrough. The driver further comprises a driving body with a driver head shaped to releasably engage the bone fastener. The driving body includes a distal opening in communication with the second bore to allow the passage of a filling composition through the second bore and through the distal opening.

### BACKGROUND

Bones in the human body sometimes undergo traumatic events. Structural damage to a bone may result from any number of traumatic events such as a fracture, tumor, or various other degenerative conditions that effect bones such as osteoporosis. As a result, a bone damaged from a traumatic event or degenerative condition may require artificial structural support for stabilization purposes. As an example, a vertebra within the spinal column may be damaged by a traumatic event. Often in such a scenario, a surgeon stabilizes the vertebra by using a driver to insert a screw into the damaged vertebral body and attach that screw to a prosthetic device such as a rod to help support and stabilize the damaged vertebra. However, sometimes it is difficult for the surgeon to achieve the required support and stabilization for the damaged vertebral body because the threads of the screw do not properly engage the vertebral bone. In some patients, an osteoporotic vertebral body may not have enough remaining bone structure to properly hold the screw.

As a result, a surgeon will use another tool, such as a syringe, to inject an adhesive material around the screw in attempt to further bond the screw with the bone. However, it is time consuming and sometimes difficult in-situ to attach a second tool, such as a syringe, to a screw. Furthermore, it may be troublesome to optimally inject adhesive material with a syringe around the screw in the precise locations where the screw requires help in being further secured to the bone. Finally, injecting cement around a screw through a syringe may pose problems for adhesive materials having higher viscosities.

Thus, systems and methods for enhancing fixation of a bone screw or other bone fixation device may be useful.

### SUMMARY

In one embodiment of the present disclosure, a driver for fastening a bone fastener to a bone comprises an elongated outer member including a first bore extending therethrough along a longitudinal axis and a coupling element that is releasably coupled with the bone fastener. The driver further comprises an elongated material conduit extending at least partially within the first bore. The material conduit including a second bore extending therethrough. The driver further comprises a driving body with a driver head shaped to releasably engage the bone fastener. The driving body includes a distal opening in communication with the second bore to allow the passage of a filling composition through the second bore and through the distal opening.

In another embodiment of the present disclosure, a system for stabilizing a bone, the system comprises a fastener including a head including a proximal opening, an elongated shaft, a first bore extending through the elongated shaft along a longitudinal axis, and an engagement member. The system further comprises a driver comprising an outer member including a second bore extending therethrough along the longitudinal axis. The outer member including a coupling element releasably couplable with the engagement member of the fastener. The driver further comprising a inner member, extending into the second bore and rotatable with respect to the outer member, including a third bore extending therethrough along the longitudinal axis and a driver head releasably couplable to the proximal opening of the fastening member. The coupling of the driver head with the proximal opening of the fastener concentrically aligns the first and third bores for passage of a filling composition therethrough.

In another exemplary aspect, the present disclosure is directed to a method for securing a fastener into a bone. The method may comprise coupling an elongated driving member to a bone fastener along a longitudinal axis, wherein the bone fastener includes a first bore in communication with at least one fenestration and the driving member includes a second bore and further wherein coupling the driving member and the bone fastener concentrically aligns the first and second bores about the longitudinal axis; rotating the bone fastener about the longitudinal axis to threadably engage the fastener with the adjacent bone; and delivering a bone filling composition into the second bore of the driving member for passage through the second bore, the first bore of the fastener, and out the at least one fenestration.

These and other aspects, forms, objects, features, and benefits of the present invention will become apparent from the following detailed drawings and description.

### BRIEF DESCRIPTION OF THE DRAWINGS

In the accompanying drawings, which are incorporated in and constitute a part of the specification, embodiments of the invention are illustrated, which, together with a general description of the invention given above, and the detailed description given below, serve to exemplify the embodiments of this invention.

FIG. 1 is side view of a segment of a lumbar spine.

FIG. 2 is a perspective view of a bone fastener according to one embodiment of the present disclosure.

FIG. 3 is a view of an exemplary driver according to one embodiment of the present disclosure.

FIG. 4 is a perspective view of the proximal portion of the driver of FIG. 3.

FIG. 5 is a cross-sectional view of the distal portion of the driver of FIG. 3.

FIG. 6 is a cross-sectional view of an alternative distal portion of the driver of FIG. 3 having an alternative bit.

FIG. 7 is an illustration of the coupling of the exemplary driver of FIG. 3 with the exemplary bone fastener of FIG. 2.

FIG. 8 is a is a partial cross-sectional view of the exemplary bone fastener of FIG. 2 coupled with the exemplary driver of FIG. 3.

FIG. 9 is an illustration of an exemplary driving tool attached to the exemplary driver of FIG. 3.

FIG. 10 is an illustration of an exemplary syringe attached to the exemplary driver of FIG. 3.

FIG. 11 is an illustration of an exemplary bone filler device inserted within the exemplary driver of FIG. 3.

FIG. 12 is a partial cross-sectional view of the exemplary bone filler device of FIG. 11 inserted within the exemplary driver of FIG. 3.

FIG. 13 is an illustration of an exemplary driver according to another embodiment of the present disclosure.

FIG. 14 is an illustration of the distal portion of the alternative driver of FIG. 13.

FIG. 15 is an illustration of an alternative bit according to another embodiment of the present disclosure.

FIG. 16 is an illustration of the engagement of the alternative driver of FIG. 13 with the exemplary bone fastener of FIG. 2.

FIG. 17 is a perspective view of another alternative driver according to one embodiment of the present disclosure.

FIG. 18 is a cross-sectional view of the driver of FIG. 17.

FIG. 19 is a partial cross-sectional view of the distal portion of the driver of FIG. 17 without a bit.

FIG. 20 is a cross-sectional view of an alternative bone fastener according to one embodiment of the present disclosure.

FIG. 21 is a perspective view of the engagement of the alternative driver of FIG. 17 with the alternative bone fastener of FIG. 20.

FIG. 22 is a cross-sectional view of the alternative driver of FIG. 17 coupled with the alternative bone fastener of FIG. 20.

FIG. 23 is a perspective view of the engagement of another alternative driver with another alternative bone fastener.

FIG. 24 is a cross-sectional view of the alternative driver of FIG. 23 coupled with the alternative bone fastener of FIG. 23.

### DETAILED DESCRIPTION

The present disclosure relates generally to the field of orthopedic surgery, and more particularly to systems and methods for securely fastening fenestrated screws within bone. For the purposes of promoting an understanding of the principles of the invention, reference will now be made to embodiments or examples illustrated in the drawings, and specific language will be used to describe these examples. It will nevertheless be understood that no limitation of the scope of the invention is thereby intended. Any alteration and further modifications in the described embodiments, and any further applications of the principles of the invention as described herein, are contemplated as would normally occur to one skilled in the art to which the disclosure relates.

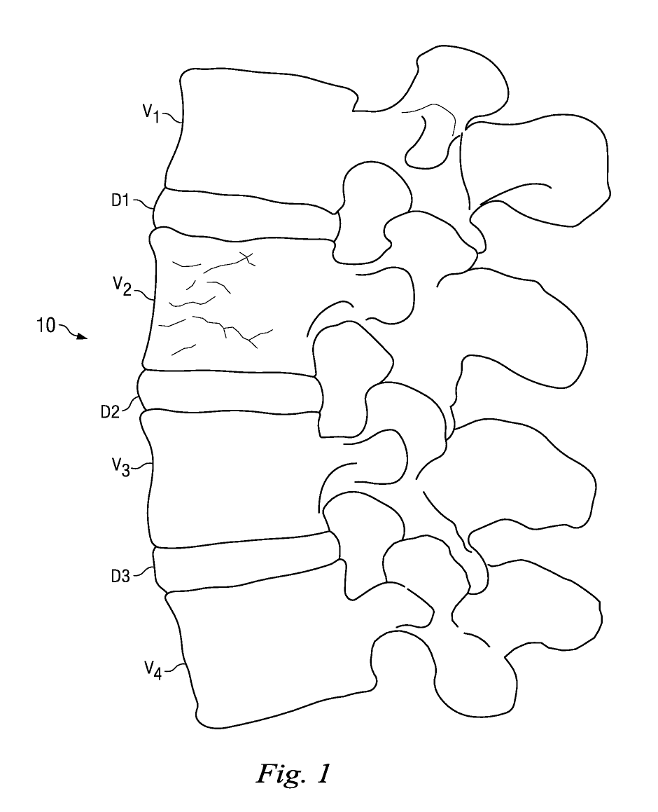

Referring first to FIG. 1, a sagittal view of a vertebral column 10 is shown, illustrating a sequence of vertebrae V1, V2, V3, V4 separated by natural intervertebral discs D1, D2, D3, respectively. Although the illustration generally depicts a lumbar section of a spinal column, it is understood that the devices, systems, and methods of this disclosure may also be applied to all regions of the vertebral column, including thoracic and cervical regions.

FIG. 2 is an illustrative embodiment of a bone fastener 100, such as bone screw, which may be used in an exemplary embodiment. Screw 100 has a an elongated body 102 along longitudinal axis L. The elongated body 102 has a proximal portion 104 and a distal portion 106. The proximal portion 104 includes a head 108. The head 108 in this exemplary embodiment is substantially spherical in shape and extends transverse to the elongated body 102. In other embodiments, the head 108 may be, but not limited to, flat, conical, balled and any other shape that may be considered by one having skill in the art. Yet in a further embodiment, head 108 may not extend transverse to longitudinal axis L.

The head 108 has a top surface 110 which provides access to a central bore 112 through proximal opening 113. Central bore 112 extends along longitudinal axis L within screw 100 from the proximal portion 104 to the distal portion 106. In addition, proximal opening 113 is shaped to correspond to the distal portion of a driver, such that the driver may engage the proximal opening 113 to drive screw 100 into a bone. In this exemplary embodiment, proximal opening 113 is torx shaped, but other configurations for proximal opening 113 may be suitable to allow the distal portion of a driver to engage the proximal opening 113.

The elongated body 102 further comprises threads 114 that help secure the screw 100 into the bone. Near the distal portion 106 of screw 100, fenestrations 116 provide window-like openings that form passageways between central bore 112 and an exterior surface 118 of screw 100. Although shown as two fenestrations within FIG. 2, fenestrations 116 are not limited to two and can be as little as one or more than two. Additionally, fenestrations may be located at the valleys of the threads (as shown) or along the projections of the threads. Furthermore, fenestrations 116 may be located anywhere along the exterior surface 118 including on opposite sides of the elongated body 102. Finally, the fenestrations 116 shown in FIG. 2 are circular in shape, but other shapes such as oval, square, and elliptical may be suitable.

The distal portion 106 of screw 100 includes tip 122. The tip 122 has a distal opening 124 that provides access to central bore 112. As will be discussed in more detail below, central bore 112 allows substances to be injected into screw 100. For example, once screw 100 has been inserted into the bone, a filling composition, such as cement, may be into the central bore 112. Upon injection, the composition may progress though central bore 112 towards distal portion 106 and may exit the bore at fenestrations 116 and the distal opening 124. Once the composition exits bore 112, it may cure, bonding screw 100 to the bone. In an alternative embodiment, screw 100 may have a closed distal end such that only fenestrations 116 provide a passageway for the composition to exit screw 100. Alternatively, the fenestrations may be omitted such that the distal opening provides the only outlet for the filling composition.

Any number of filling compositions may be injected by a driver into screw 100. Examples of suitable filling compositions that may be injected into screw 100 include bone cements such as those made from polymethylmethacrylate (PMMA), calcium phosphate, hyrdroxyapatite-tricalcium phosphate (HA-TCP) compounds, bioactive glasses, polymerizable matrix comprising a bisphenol-A dimethacrylate, or CORTOSS™ by Orthovita of Malvern, Pa. (generically referred to as a thermoset cortical bone void filler). Calcium sulfate bone void fillers and other filling compositions or combinations of filling compositions may also be used. Bone void fillers or bone cements may be treated with biological additives such as demineralized bone matrix, collagen, gelatin, polysaccharide, hyaluronic acid, keratin, albumin, fibrin, cells and/or growth factors. Additionally or alternatively, bone void fillers or bone cements may be mixed with inorganic particles such as hydroxyapatite, fluorapatite, oxyapatite, wollastonite, anorthite, calcium fluoride, agrellite, devitrite, canasite, phlogopite, monetite, brushite, octocalcium phosphate, whitlockite, tetracalcium phosphate, cordierite, berlinite or mixtures thereof.

Other osteoinductive, osteoconductive, or carrier materials that may be injected, extruded, inserted, or deposited into vertebral bone may include collagen, fibrin, albumin, karatin, silk, elastin, demineralized bone matrix, or particulate bone. Various bone growth promoting biologic materials may also be added to the bone filler including mysenchymal stem cells, hormones, growth factors such as transforming growth factor beta (TGFb) proteins, bone morphogenic proteins (including BMP and BMP2), or platelet derived growth factors. The above listings of filling compositions that may be used in the embodiments of this disclosure are for exemplary purposes and are not to be construed as limitations.

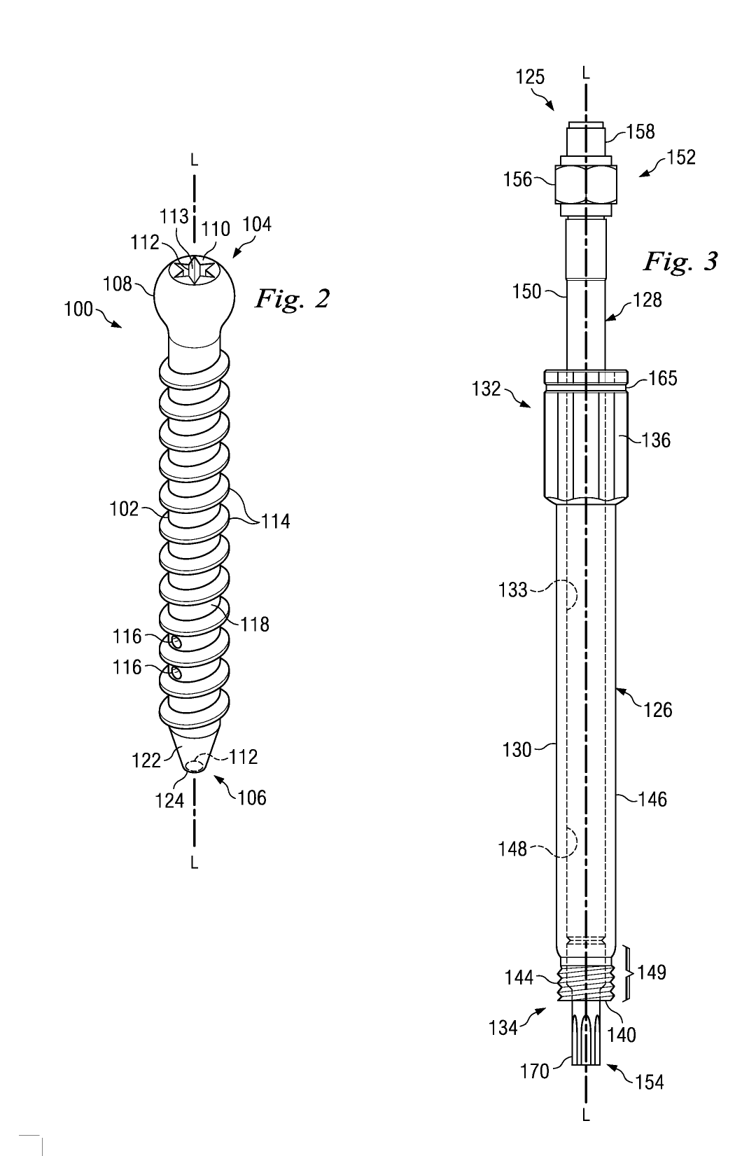

Referring now to FIG. 3, an exemplary driver system 125 is shown. Driver system 125 is configured to engage a bone fastener, such as screw 100, to fasten the bone fastener to bone, and to provide access to inject filling compositions, such as those described above, into the bone fastener.

Driver 125 includes a sleeve 126 and an adapter 128. The sleeve 126 has an elongated body 130 along longitudinal axis L. The elongated body 130 is generally cylindrical in shape, but other cross-sectional shapes including triangular, square, hexagonal, elliptical, and tapered, may be suitable. The elongated body 130 has a has an exterior surface 146, an interior surface 148, a proximal portion 132, and a distal portion 134. The proximal portion 132 includes a grip 136 that is used by a surgeon to manipulate driver 125. The distal portion 134 of sleeve 126 has a section 149 that tapers towards longitudinal axis L forming a conical shape end for sleeve 126. The tapered section 149 has a threaded area 144. In alternative embodiments, the threaded area may not be tapered.

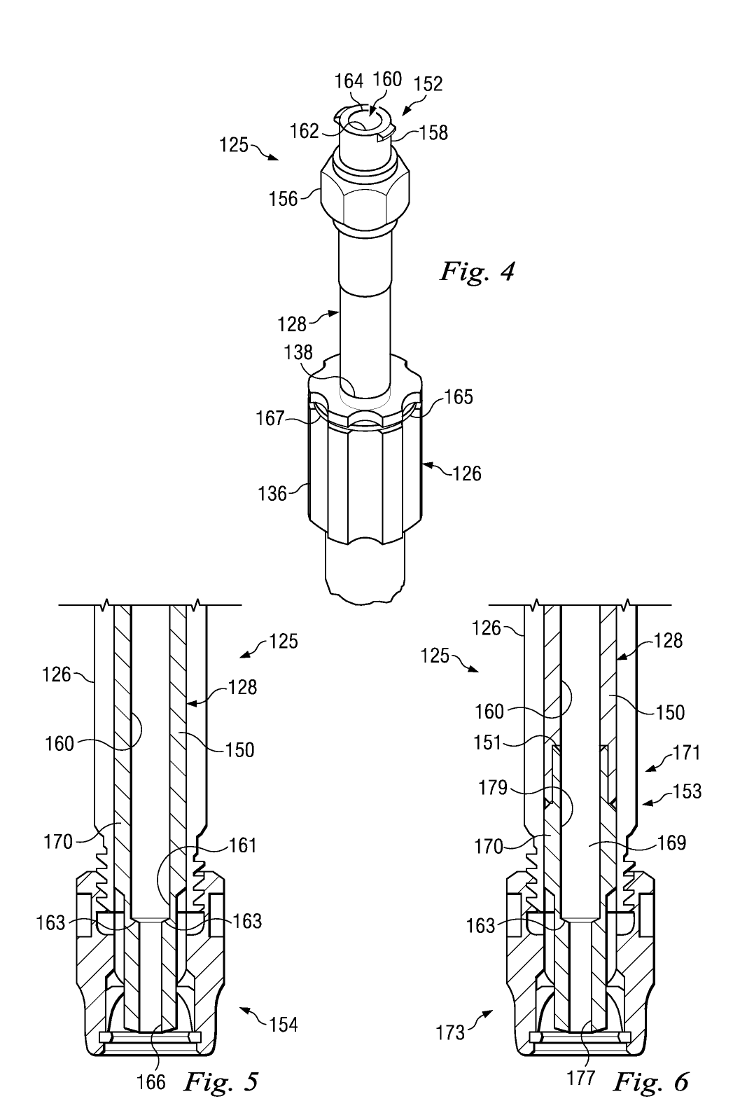

Sleeve 126 further includes a central bore 133 that extends along longitudinal axis L from the proximal portion 132 to the distal portion 134. The central bore 133 is defined by the interior surface 148 of the sleeve 126. Additionally, the sleeve has a proximal opening 138 (see FIG. 4) and a distal opening 140 that provide access to central bore 133. Near the proximal portion 132 of sleeve 126 that contains grip 136, central bore 133 may taper transversely away from longitudinal axis L such that the central bore 133 has a larger diameter in the proximal portion 132 than in the distal portion 134 of sleeve 126. In alternative embodiments, the central bore 133 may have a uniform diameter along longitudinal axis L.

Also shown in FIG. 3 is adapter 128 having an elongated body 150 along longitudinal axis L. The elongated body 150 has a proximal portion 152 and a distal portion 154. The elongated body 150 is generally cylindrical in shape, but other cross-sectional shapes may be suitable including triangular, square, hexagonal, and elliptical. Regardless of the cross-sectional shape of adapter 128, it is configured to be inserted into the proximal opening 138 of central bore 133 and extend along longitudinal axis L through distal opening 140. Upon insertion of adapter 128 within central bore 133, at least a part of the proximal portion 152 and distal portion 154 of adapter 128 extend beyond central bore 133 along longitudinal axis L.

Adapter 128 further includes a central bore 160 (FIGS. 4-6, 8), which may be a material conduit, that extends along longitudinal axis L from the proximal portion 152 to the distal portion 154. The adapter 128 further includes a proximal opening 162 (FIG. 4) on the top surface 164 of the proximal portion 152. Additionally, the adapter 128 has a distal opening 166 (FIG. 5) on the bottom surface 168 of the distal portion 154. Proximal opening 162 and distal opening 166 provide access to central bore 160.

As shown in greater detail in FIG. 4, the part of the proximal portion 152 of adapter 128 that extends proximally beyond central bore 133 includes a driving tool engagement interface 156. Driving tool engagement interface 156 provides an interface for a driving tool, such as a wrench, screw driver, handle, drill, and any other tool one skilled in the art may use to manipulate driver 125. The driving tool engagement interface 156 within the exemplary embodiment is hexagonal in shape, but any other shape that mates with an appropriate driving tool may be suitable. A driving tool mated with driving tool engagement interface 156 can rotate adapter 128 about longitudinal axis L.

The adapter 128 further includes a delivery system interface 158 located on the proximal portion 152 of adapter 128 that extends beyond central bore 133. The delivery system interface 158 allows a delivery system (not shown) to be attached to adapter 128 to be able to access central bore 160. For example, the delivery system may include a syringe, a pump, or other viscous material advancement systems for high or low pressure material delivery. The delivery system interface 158 may be a luer connection, a threaded connection, or any other connection known in the art.

Surrounding at least a section of the proximal portion 152 of the adapter 128 housed within central bore 133 is an annular flange 165. The annular flange 165 is located within the portion of the central bore 133 that tapers transversely away from longitudinal axis L. Annular flange 165 extends transversely from longitudinal axis L such that an edge 167 is in close proximity to the interior surface 148 of sleeve 126, but not touching while adapter 128 is aligned along the longitudinal axis L. The annular flange 165 limits the movement of adapter 128 away from longitudinal axis L while adapter 128 is being rotating with a driving tool. Specifically, edge 167 contacts the interior surface 148 when adapter 128 is rotated too far offline from longitudinal axis L. In an alternative embodiment, the annular flange 165 may be formed as a integrated component of adapter 128.

Referring again to FIG. 3, the distal portion 154 of the adapter 128 includes a bit 170. Specifically, bit 170 extends along longitudinal axis L beyond the central bore 133 of the sleeve 126. The bit 170 is configured to engage the proximal opening 113 of screw 100. The engagement of bit 170 with proximal opening 113 of screw 100 enables the adapter 128 to drive screw 100 into a bone. Bit 170 shown in FIG. 3 has a torx shaped tip, although other configurations for bit 170 may be utilized to engage the proximal opening 113 of screw 100. There is no implied limitation that bit 170 have a torx shaped tip and other shaped tips as may be known to one skilled in the art may be used for bit 170.

FIG. 5 shows a cross-sectional view of the distal portion 154 of adapter 128. In this embodiment, bit 170 may be formed as part of the elongated body 150 of the adapter 128. An interior surface 161 of elongated body 150 tapers towards longitudinal axis L within the bit 170 portion of central bore 160 to form stops 163. The stops 163 may be used to prevent certain tools inserted within the central bore 160 from exiting distal opening 166. Additionally, the tapering of interior surface 161 narrows the diameter of bore 160 with respect to the diameter of the central bore 160 housed proximally to the tapered interior surface.

FIG. 6 shows a cross-sectional view of an alternative embodiment for the distal portion 154 of adapter 128. As seen in this alternative embodiment, bit 170 may be a separate component from elongated body 150 of the adapter 128. As a separate component, bit 170 has a central bore 169 extending longitudinally along longitudinal axis L from a proximal portion 171 to a distal portion 173. In such an alternative embodiment, the distal portion 153 of elongated body 150 may have a recessed opening 151 that receives the corresponding proximal portion 171 of bit 170 such that proximal portion 171 is push-fit into recessed opening 151. Other connection methods between bit 170 and elongated body 150 may be used such as snap fit, sonic welding, threaded connection, or any other method that may be used by one having skill in the art to join bit 170 to elongated body 150. Additionally, interior surface 179 of bit 170 tapers towards longitudinal axis L near the distal portion 173 to form stops 163. Finally, in this alternative embodiment bit 170 has a distal opening 177 to allow access to central bore 169. The stops 163 may be used to prevent tools inserted within adapter 128 from exiting distal opening 177 in this alternative embodiment. This alternative embodiment, with modular drill bits, may allow the drill bits to be removed and exchanged.

All of the embodiments disclosed herein in whole or in part may be constructed of biocompatible materials of various types including metals or polymers. Examples of materials include, but are not limited to, non-cobalt-chromium alloys, titanium alloys, nickel titanium alloys, and/or stainless steel alloys, any member of the polyaryletherketone (PAEK) family such as polyetheretherketone (PEEK), carbon-reinforced PEEK, or polyetherketoneketone (PEKK); polysulfone; polyetherimide; polyimide; ultra-high molecular weight polyethylene (UHMWPE); and/or cross-linked UHMWPE. In one exemplary embodiment the adapter 128 may be formed all or in part of a metal and the sleeve 126 may be formed all or in part of a polymer.

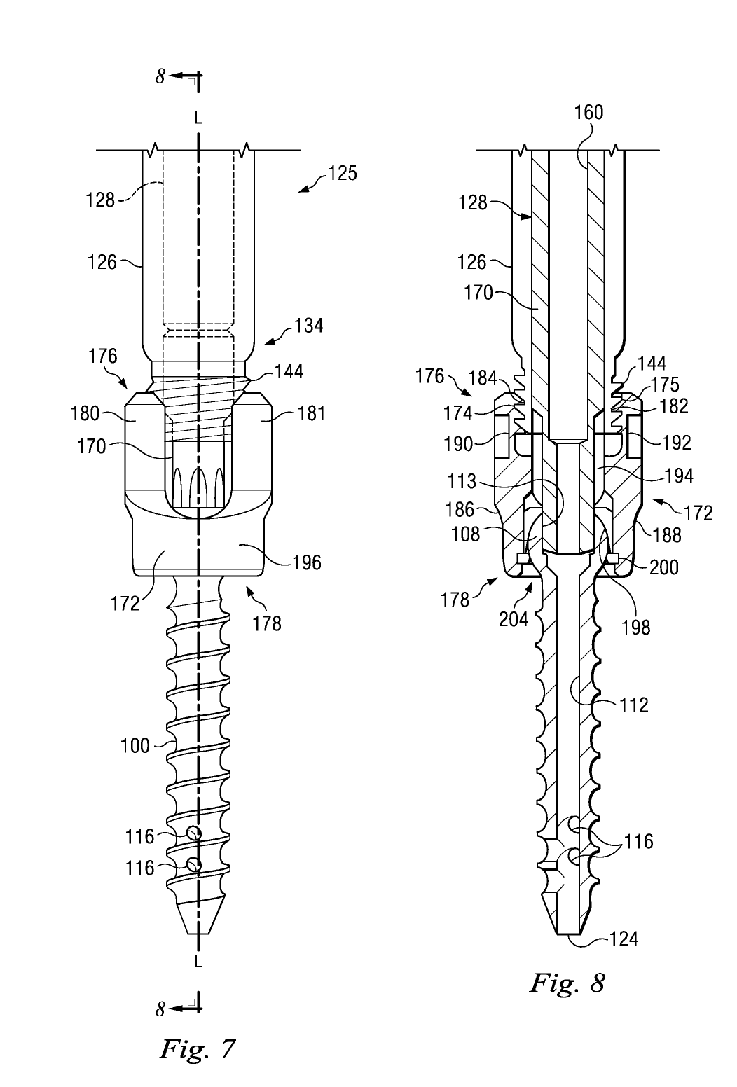

FIGS. 7 and 8 show screw 100 engaged with driver 125. As shown, bone screw 100 has been assembled with a multi-axial engagement member 172 that surround the head 108 and may be pivotable and rotatable with respect to the head. The multi-axial engagement member 172 may be considered a component of the bone fastener 100. The multi-axial engagement member 172 may provide an engagement interface between driver 125 and screw 100. Furthermore, multi-axial engagement member 172 may help stabilize the screw 100 with respect to driver 125 during the driving and injecting of screw 100.

The multi-axial engagement member 172 has a proximal portion 176 and a distal portion 178. Extending along the longitudinal axis L from the proximal portion 176 to the distal portion 178 is central bore 194. The proximal portion 176 consists of tab members 180, 181 that extend longitudinally with respect to longitudinal axis L. The tab members each have an inner surface 182, 184 that include threaded portions 174, 175. Threaded portions 174, 175 threadedly engage threaded areas 144 of the distal portion of sleeve 126. The inner surfaces 182, 184 further define the central bore 194 extending longitudinally along the longitudinal axis L in the proximal portion 172. The outer surface 186, 188 of tab members 180, 181 have an indentation 190, 192 respectively for allowing alternative embodiments of sleeve 126 having tab projections to engage the multi-axial engagement member 172 via the indentations 190 and 192.

The distal portion 178 of the multi-axial engagement member 172 includes a base 196 that supports tab members 180, 181 respectively. The inner surface 198 of base 196 forms the distal portion of central bore 194. The inner surface 198 may be concave or spherically shaped. It should be noted in other embodiments that inner surface 198 may be flat, tapered, or any other shape that one skilled in the art may utilize to correspond to the shape of head 108 of screw 100.

The portion of central bore 194 defined by inner surface 198 houses the head 108 of screw 100. Because the head 108 in this exemplary embodiment is substantially spherical to correspond to spherically shaped inner surface 198, head 108 can articulate with respect to bore 194. An insert 200 may be housed within the base 196 adjacent a distal opening 204 for central bore 194. The insert 200 may be circular, C-shaped, or any other shape that one skilled in the art may utilize. The insert 200 interacts with head 108 to further help the head 108 articulate with respect to multi-axial engagement member 172 and to prevent dislocation of screw 100 from multi-axial engagement member 172.

In the exemplary embodiment shown in FIGS. 7 and 8, sleeve 126 may be engaged with screw 100 using the multi-axial engagement member 172. For engagement purposes, the sleeve 126, adapter 128, engagement member 172, and screw 100 are aligned along longitudinal axis L. Sleeve 126 is inserted between tab members 180 and 181 such that the threaded areas 144 on the distal portion 134 of sleeve 126 are aligned with the threaded portions 174, 175 of tab members 180, 181 respectively. The sleeve 126 may be rotated clockwise such that threaded areas 144 threadedly engage threaded portions 174, 175. Through the sleeve 126, adapter 128 may be lowered until the bit 170 is removably engaged with screw 100.

Upon engagement of bit 170 into the proximal opening 113 of screw 100, the central bores 112 and 160 of screw 100 and adapter 128 respectively are concentrically aligned to form a continuous bore extending from the proximal opening 162 of the adapter 128 to the distal opening 124 of the screw 100. Additionally, it should be noted that once the bit 170 is engaged with screw 100 a seal may be formed such that any substance progressing through the concentrically aligned bores cannot escape between the bit 170 and screw 100.

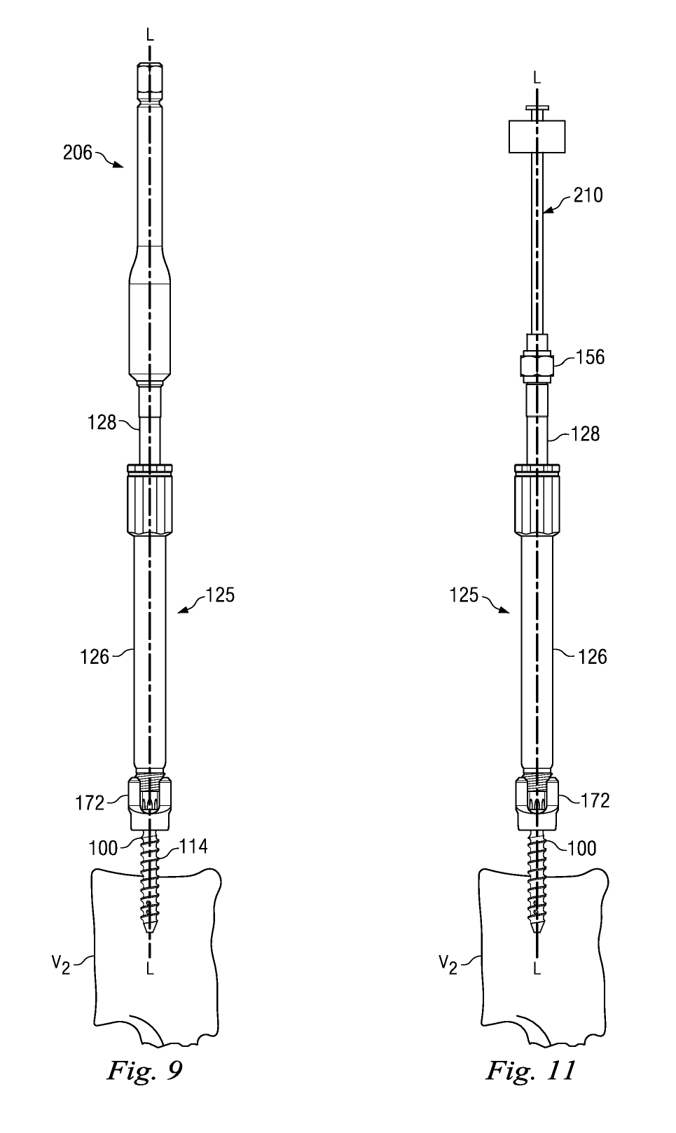

Engagement of bit 170 into the corresponding proximal opening 113 of the screw 100, enables the adapter 128 to be used to drive the screw 100 into bone. As shown in FIG. 9, a driving tool 206 is attached to driver 125 via the driving tool engagement interface 156 (see FIG. 3). As previously mentioned, driving tool engagement interface 156 provides an interface for a driving tool 206, such as a wrench, screw driver, handle, drill, and any other tool one skilled in the art may use with driver 125. The driving tool 206 engaged with driving tool engagement interface 156 can rotate adapter 128 about longitudinal axis L. By rotating adapter 128, bit 170 inserted into the proximal opening 113 of screw 100 in turn rotates the screw 100. As shown in FIG. 9, rotating screw 100 causes threads 114 to engage the vertebral body V2 such that screw 100 may be secured to the vertebral bone. Therefore, driving tool 206 may be used to drive screw 100 into V2 by rotating adapter 128 of driver 125 about longitudinal axis L.

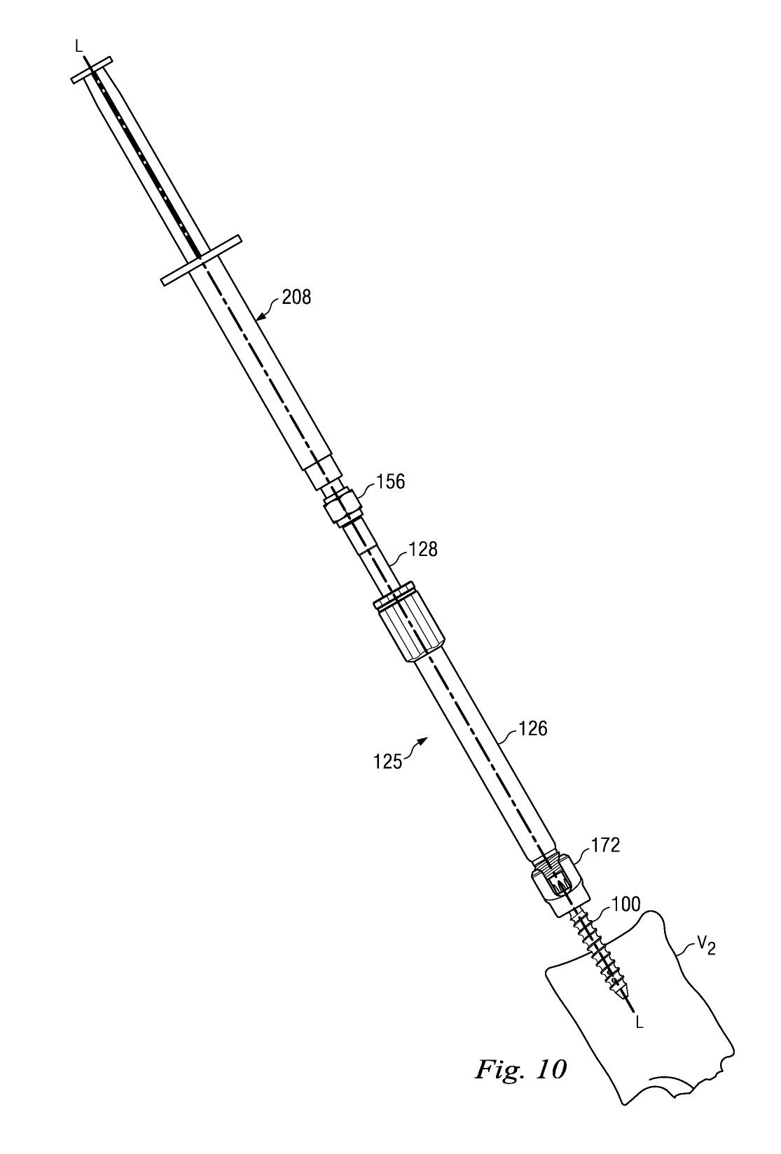

During the driving of screw 100 into V2 by driver 125, bone particles may enter and block fenestrations 116 and/or the distal opening 124 of central bore 112 of the screw 100. If the fenestrations 116 and/or distal opening 124 are blocked by bone particles then a substance injected into central bore 112 of the screw 100 may not be able to exit central bore 112. To alleviate any potential blockage, as shown in FIG. 10, once screw 100 is driven into V2 the driving tool 206 is detached from driver 125 and may be replaced by a syringe 208. The syringe 208 is attached to driver 125 via delivery system interface 158 (see FIG. 3). The syringe 208 may be filled with a flushing material, such as saline, so that injection of the flushing material traverses the central bore 160 of the adapter 128 and into the central bore 112 of the screw 100. The injection of the flushing material removes any bone particles that may be blocking fenestrations 116 and/or distal opening 124 of the screw 100. Thus, the use of the syringe 208 helps alleviate any bone particles blocking fenestrations 116 and/or distal opening 124 of the screw 100 to allow a subsequent substance injected into central bores 160 and 112 to be able to exit the fenestrations 116 and the distal opening 124 of the screw 100. In an alternative embodiment, a syringe may also be used for, but not limited to, injecting a barium tracer and any other filling composition discussed with respect to driver 125 into screw 100.

As shown in FIG. 11, after screw 100 is at least partially driven by driver 125 into V2 and bone particles blocking fenestrations 116 and/or distal opening 124 of screw 100 have been alleviated, a material delivery system such as a bone filler device 210 may be inserted through the proximal opening 162 of the adapter 128 and the central bore 160. The bone filler device 210 attached to the driver 125 may inject a filling composition such as cement through driver 125 into screw 100. The rigidity of driver 125 may be particularly suitable for high pressure injection of materials. For example, as compared to lower pressure systems such as syringe-only systems, a cement of higher viscosity may be injected through adapter 128 into screw 100

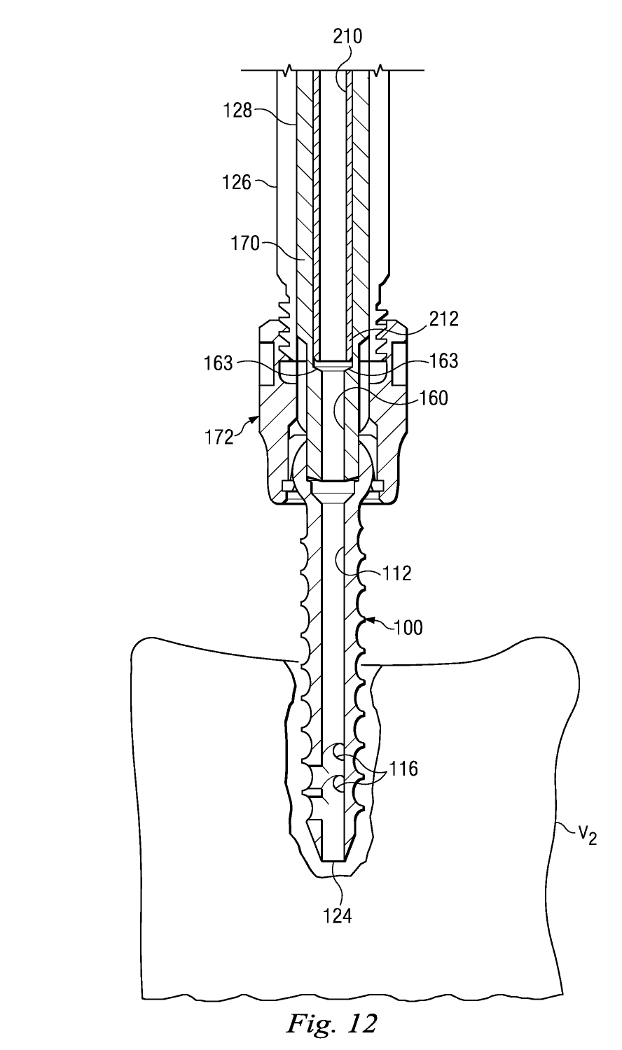

As can be further seen in FIG. 12, bone filler device 210 is inserted through central bore 160 until the distal portion 212 abuts stops 163. The bone filler device 210 abutted against stops 163 may be used to deliver a filling composition such as cement into the distal portion of central bore 160 which can then flow into central bore 112 of the screw 100. Because of fenestrations 116 and distal opening 124 of the screw 100, the composition is allowed to exit central bore 112. As the cement passes out of the central bore 112, the cement may engage the various pores, concavities and interstices of the vertebral body V2, thereby creating a mass or collection of cement about the screw 100. After curing, the cement creates a firm fixation or anchoring of the screw 100 in the vertebral body V2 or any other bone structure. Additionally, since the cement tends to engage the various pores, concavities and interstices of a bone, such as V2, the bone may tend to be strengthened by the infusion of cement through and around screw 100. Thus, the combination of driver 125 and screw 100 enables a system and method for securely anchoring screw 100 into bone to provide structural support and stabilization for damaged bones.

It should be noted that driver 125 and screw 100 may be decoupled from one another after driving and injection. Specifically, the threaded areas 144 of sleeve 126 are disengaged from the threaded portions 174 and 175 of the multi-axial engagement member 172. When the threaded areas 144 of sleeve 126 are disengaged from the threaded portions 174 and 175 of the multi-axial engagement member 172 the driver 125 may be removed from screw 100.

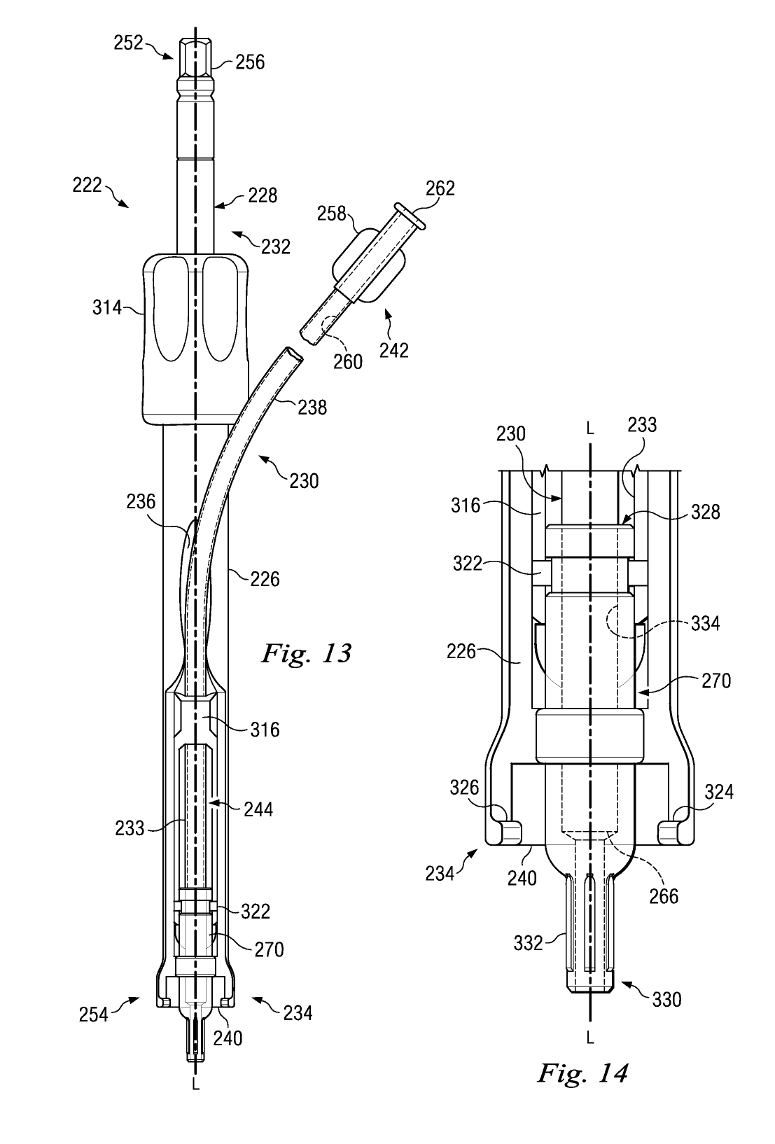

FIG. 13 shows an alternative embodiment of a driver labeled by reference numeral 222. Driver 222 is composed of sleeve 226, adapter 228, and a material conduit 230. The sleeve 226 has an elongated body along longitudinal axis L. The elongated body of sleeve 226 is generally cylindrical in shape, but other cross-sectional shapes may be suitable including, triangular, square, hexagonal, elliptical, and tapered. Furthermore, sleeve 226 has a distal opening 240 that provides access to a central bore 233 that extends along longitudinal axis L through the length of sleeve 226. Regardless of the cross-sectional shape of sleeve 226, it is configured to receive adapter 228 along longitudinal axis L within central bore 233. In addition, sleeve 226 has an aperture 236 on the exterior surface that provides access to central bore 233.

Additionally, sleeve 226 has a proximal portion 232 and a distal portion 234. The proximal portion 232 of sleeve 226 includes a thumbwheel 314. Thumbwheel 314 provides a mechanism to translate the adapter 228 along longitudinal axis L within the sleeve 226. Specifically, thumbwheel 314 may be rotated about longitudinal axis L such that rotation translates adapter 228 along longitudinal axis L towards the distal portion 234 of sleeve 226. As an alternative mechanism, a scroll wheel may be used that enables one to scroll the adapter 228 along longitudinal axis L towards the distal portion 234 of sleeve 226. Other mechanisms may be used to translate adapter 228 along longitudinal axis L relative to sleeve 226 as may be known to one skilled in the art.

FIG. 14 shows the distal portion 234 of sleeve 226 in greater detail. Specifically tabs 324 and 326 projecting around the distal opening 240 are shown. Tabs 324 and 326 are configured to engage with indentations 190 and 192 of the multi-axial engagement member 172 (see FIG. 16). Therefore, tabs 324 and 326 allow for screw 100 having a multi-axial engagement member 172 to be attached to the distal portion 234 of sleeve 226. Furthermore, the engagement of tabs 324 and 326 with indentations 190 and 192 help stabilize the screw 100 with respect to driver 222 during the driving and injecting of screw 100.

As previously mentioned, adapter 228 is shown within FIG. 13. The adapter 228 has an elongated body along longitudinal axis L. The elongated body of adapter 228 is generally cylindrical in shape, but other cross-sectional shapes are considered, but not limited to triangular, square, hexagonal, elliptical, and tapered. Adapter 228 has a proximal portion 252 and a distal portion 254.

The proximal portion 252 of adapter 228 includes a driving tool engagement interface 256. Driving tool engagement interface 256 provides an interface for a driving tool, such as a wrench, screw driver, handle, drill, and any other tool one skilled in the art may use with driver 222. A driving tool engaged with driving tool engagement interface 256 may be used to rotate driver 222 about longitudinal axis L.

As shown in FIGS. 13 and 14, the distal portion 254 of adapter 228 includes a housing 316 that is located within central bore 233 of sleeve 226. As will be discussed in more detail below, the housing 316 is designed to secure a bit portion 270 of material conduit 230 to adapter 228.

As previously mentioned, FIG. 13 also shows the material conduit 230. The material conduit 230 has a flexible elongated body 238. The elongated body 238 is generally cylindrical in shape, but other cross-sectional shapes are considered, but not limited to triangular, square, hexagonal, elliptical, and tapered. Furthermore, material conduit 230 has a proximal opening 262 and distal opening 266 that provides access to a central bore 260 that extends through the length of material conduit 230.

The material conduit 230 has a proximal portion 242 and a distal portion 244. The proximal portion 242 extends outward from the central bore 233 of the sleeve 226 through aperture 236. The proximal portion 242 includes a delivery system interface 258. The delivery system interface 258 allows a delivery system (not shown) to be attached to material conduit 230 to be able to access central bore 260. For example, the delivery system may include a syringe and/or bone filler device to be connected to material conduit 230 in order to access central bore 260. The delivery system interface 258 may be a luer connection, a threaded connection, or any other connection known in the art.

The distal portion 244 of material conduit 230 extends through aperture 236 of sleeve 226 into bore 233. The distal portion 244 of material conduit 230 includes the bit 270. Bit 270 has a proximal portion 328 and a distal portion 330. Specifically, the proximal portion 328 of bit 270 is secured by engagement mechanism 322 of housing 316 to adapter 228.

The distal portion 330 of bit 270 contains a torx shaped tip 332. Although bit 270 shown in FIG. 14 has a torx shaped tip 332, other configurations for the tip may be utilized to engage the proximal opening 113 of screw 100. Additionally, bit 270 has a central bore 334 extending along longitudinal axis L through the entire longitudinal length of bit 270. Furthermore, bit 270 has a proximal opening and a distal opening to allow access to central bore 334.

In an alternative embodiment, bit 270 may be a separate component from material conduit 230. In such an embodiment, the distal portion 244 of the material conduit includes a coupling element which provides a push-fit interface for releasably coupling the material conduit 230 with bit 270. Specifically, the coupling element is push-fit into central bore 334 of bit 270 to form a seal between material conduit 230 and bit 270. In other embodiments, the coupling element of material conduit 230 may be coupled with bit 270 by threaded connections, snap-fit, sonic welding, and any other method that one skilled in the art may utilize.

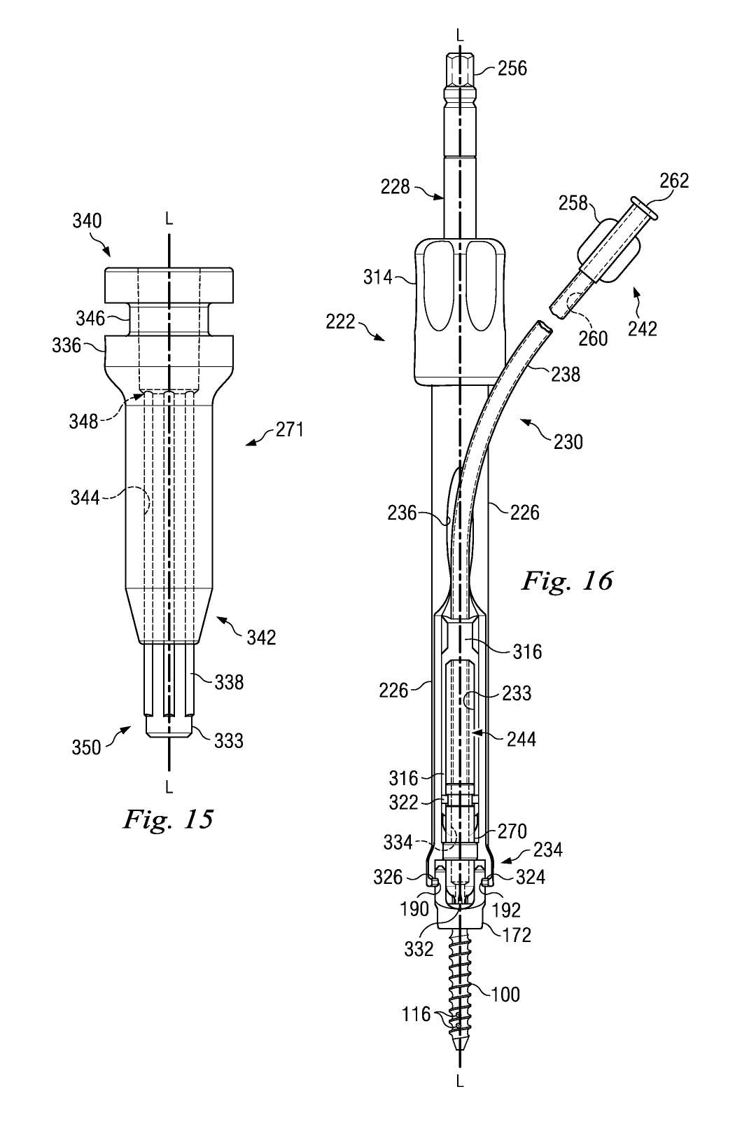

FIG. 15 shows an alternative bit 271 that may be connected to the distal portion 244 of the material conduit 230 in place of bit 270. Bit 271 is secured to adapter 228 via engagement mechanism 322 (see FIG. 14) of housing 316. Bit 271 is comprised of a sleeve 336 and an elongated body 338. Sleeve 336 has a proximal portion 340 and a distal portion 342. Central bore 344 extends through sleeve 336 along longitudinal axis L. The proximal portion 340 of sleeve 336 includes an engagement mechanism interface 346 that engages engagement mechanism 322 to secure the bit 271 to housing 316.

Elongated body 338 has a proximal portion 348 and distal portion 350. The elongated body 338 has a central bore extending therethrough along longitudinal axis L. Additionally, the elongated body 338 has a proximal opening and a distal opening to allow access to the central bore of the elongated body 338. Furthermore, the elongated body 338 has a tip 333. The tip 333 has a cross-sectional torx shape, but other cross-sectional shapes are considered. Tip 333 is configured to engage the proximal opening 113 of screw 100 in order to drive screw 100 into a bone.

Elongated body 338 is positioned within central bore 344 of sleeve 336 such that the distal portion 350 extends beyond central bore 334 along longitudinal axis L. To obtain such positioning, sleeve 336 many be molded over elongated body 338. However, in other embodiments elongated body 338 may be positioned within sleeve 336 by push-fit, snap fit, sonic welding, and any other method that one skilled in the art may utilize.

It should be noted that sleeve 336 and elongated body 338 may be composed of the same and/or different biocompatible materials. For example, sleeve 336 and elongated body 338 may be composed of metals such as cobalt-chromium alloys, titanium alloys, nickel titanium alloys, and/or stainless steel alloys. Additionally, sleeve 336 and elongated body 338 may be composed of plastics such as any member of the polyaryletherketone (PAEK) family such as polyetheretherketone (PEEK), carbon-reinforced PEEK, or polyetherketoneketone (PEKK); polysulfone; polyetherimide; polyimide; ultra-high molecular weight polyethylene (UHMWPE); and/or cross-linked UHMWPE.

It may be advantageous in one embodiment of bit 271 for sleeve 336 to be comprised of plastic and elongated member 338 to be comprised of metal. In such an embodiment, a plastic sleeve 336 allows for a more conducive fit for bit 217 within housing 316 while a metal tip 333 still allows bit 271 to have enough rigidity to drive screw 100 into a bone.

In an alternative embodiment, bit 271 may be a separate component from material conduit 230. In such a scenario the distal portion 244 of the material conduit 230 includes a coupling element. The coupling element provides a push-fit interface for releasably coupling the material conduit 230 with bit 271. Specifically, coupling element is push-fit into central bore 344 of bit 270 to form a seal between material conduit 230 and bit 271. In other embodiments, the coupling element of material conduit 230 may be coupled with bit 271 by threaded connections, snap-fit, sonic welding, and any other method that one skilled in the art may utilize.

All of the embodiments disclosed herein in whole or in part may be constructed of biocompatible materials of various types including metals or polymers. Examples of materials include, but are not limited to, non-cobalt-chromium alloys, titanium alloys, nickel titanium alloys, and/or stainless steel alloys, any member of the polyaryletherketone (PAEK) family such as polyetheretherketone (PEEK), carbon-reinforced PEEK, or polyetherketoneketone (PEKK); polysulfone; polyetherimide; polyimide; ultra-high molecular weight polyethylene (UHMWPE); and/or cross-linked UHMWPE. In one exemplary embodiment the adapter 228 may be formed all or in part of a metal and the sleeve 226 may be formed all or in part of a polymer.

FIG. 16 shows the screw 100 engaged with the driver 222. Specifically, indentations 190 and 192 of multi-axial engagement member 172 are configured to engage with tabs 324 and 326 of sleeve 226 to couple screw 100 with driver 222 along longitudinal axis L.

Additionally shown in FIG. 16, thumbwheel 314 has been manipulated to translate adapter 228 along longitudinal axis L towards the distal portion 234 of sleeve 226. Because bit 270 is secured to adapter 228 via engagement mechanism 322, translation of adapter 228 also translates bit 270 along longitudinal axis L. As shown in FIG. 16, adapter 228 has been translated along longitudinal axis L such that bit 270 is inserted into the proximal opening 113 of screw 100. Specifically, tip 332 of bit 270 extends into the proximal opening 113 of the screw 100. The extension of tip 332 into the proximal opening 113 of screw 100 may form a seal between bit 270 and screw 100. Upon insertion of tip 332 into the proximal opening 113 of screw 100, the central bores 260, 334, and 112 of material conduit 230, bit 270, and screw 100 respectively are concentrically aligned to form a continuous bore extending from the proximal opening 262 of the material conduit 230 to the distal opening 124 of the screw 100. Thus, the engagement of tip 332 with the proximal opening 113 allows driver 222 to be used to both drive screw 100 into a bone and provide access via the distal opening of tip 332 into central bore 112 in order to inject screw 100 with filling composition.

As previously mentioned, driver 222 as shown in FIG. 16 can be used to drive the screw 100 into a bone. For example, a driving tool 206, as previously described, may be movably attached to driving tool engagement interface 256, such that driving tool 206 rotates driver 222 about longitudinal axis L. Rotation of driver 222 causes bit 270 to rotate screw 100 into a bone. Additionally, a syringe 208, filled with flushing material, may be attached to material conduit 230 via delivery system interface 258 to inject flushing material through central bores 260, 334, and 112. The injection of the flushing material removes any bone particles that may be blocking fenestrations 116 and/or distal opening 124 of the screw 100.

Finally, a material delivery system such as a bone filler device may be attached to the proximal opening 262 of the material conduit 230 to provide fluid communication with central bore 260. In an alternative embodiment, a material delivery system may be inserted into to the delivery system interface 258. It should be noted that any number of filling compositions such as those listed above may be injected by driver 222 into screw 100.

A bone filler device attached to the driver 222 via material conduit 230 is used to inject a filling composition such as cement through driver 222 via central bores 260 and 334 into central bore 112 of screw 100. As previously discussed, fenestrations 116 and distal opening 124 of the screw 100 allow the filling composition to exit central bore 112. As the composition passes out of the central bore 112, the filling composition may engage the various pores, concavities and interstices of the bone structures surrounding screw 100, thereby creating a mass or collection of filling composition about the screw 100. After curing, the filling composition creates a firm fixation or anchoring of the screw 100 in a bone structure. Additionally, since the filling composition may tend to engage the various pores, concavities and interstices of a bone, the bone may tend to be strengthened by the infusion of filling composition through and around screw 100. Thus, driver 222 enables a system and method for securely anchoring screw 100 into bone to provide structural support and stabilization for damaged bones.

It should be noted that driver 222 and screw 100 may be decoupled from one another after driving and injection. Specifically, the tabs 324 and 326 of sleeve 226 are disengaged from the indentations 190 and 192 of multi-axial engagement member 172. When the tabs 324 and 326 of sleeve 226 are disengaged from the indentations 190 and 192 of multi-axial engagement member 172 the driver 222 may be removed from screw 100.

In an alternative embodiment of a driver similar to driver 222, the adapter may be cannular to serve as the material conduit. In such an alternative embodiment, bit connects to the distal end of the adapter. In this alternative, the adapter may further includes a delivery system interface located on the proximal portion of the adapter. The delivery system interface allows a delivery system to be attached to adapter to be able to access the central bore. The elongated body of the adapter may be configured to be received within the central bore of the bit and may form a seal between the adapter and the bit.

In this alternative embodiment, a thumbwheel may be manipulated to translate adapter along longitudinal axis L. Because the bit is secured to the adapter, translation of adapter also translates the bit along longitudinal axis L until the bit extends into the proximal opening 113 of the screw 100. Upon insertion of the bit tip into the proximal opening 113 of screw 100, the central bores of the adapter, the bit, and the screw 100 respectively are concentrically aligned to form a continuous bore extending from the proximal opening of the adapter to the distal opening of the screw 100. Thus, this alternative embodiment of the driver with a cannular adapter may be used to both drive screw 100 into a bone and provide access via the central bores of adapter and the bit into central bore 112 in order to inject screw 100 with filling composition.

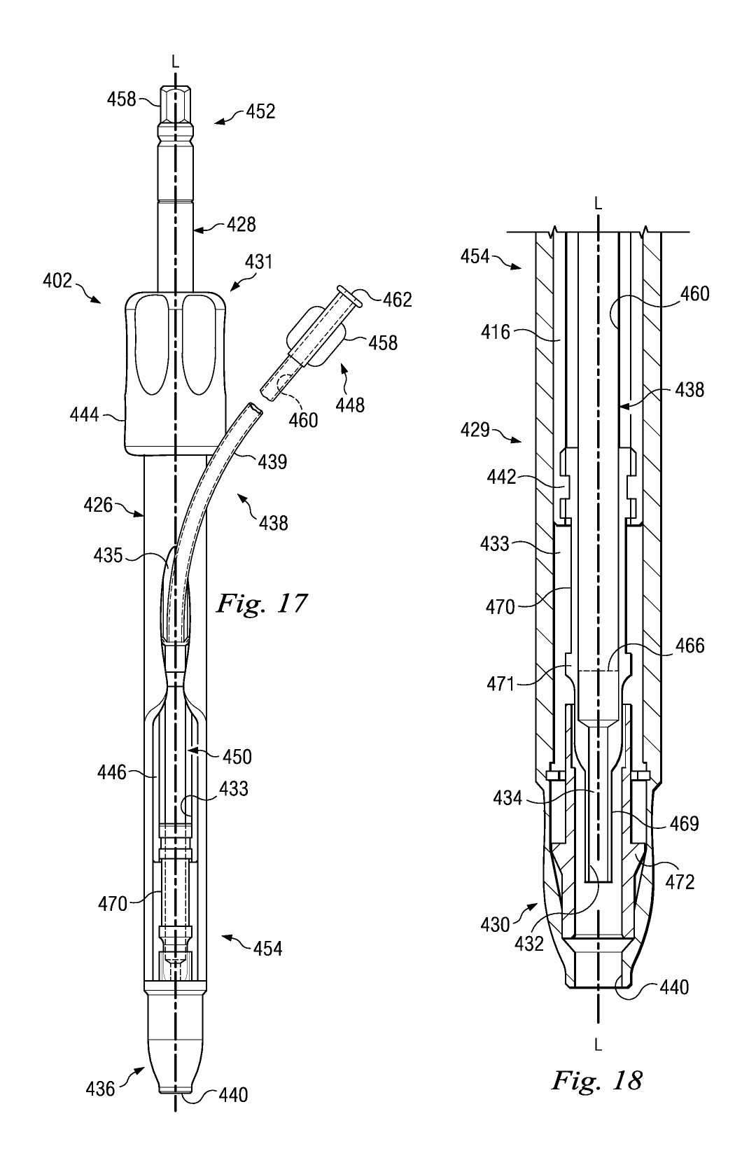

FIGS. 17 and 18 show an alternative embodiment of a driver labeled by reference numeral 402. Driver 402 is composed of sleeve 426, adapter 428, and a material conduit 438. The sleeve 426 has an elongated body along longitudinal axis L. The elongated body of sleeve 426 is generally cylindrical in shape, but other cross-sectional shapes are considered, but not limited to triangular, square, hexagonal, elliptical, and tapered.

Sleeve 426 has a proximal portion 431 and a distal portion 436. Furthermore, sleeve 426 includes a central bore 433 that extends along longitudinal axis L through the length of sleeve 426. Specifically, the central bore 433 of sleeve 426 is designed to receive adapter 428. The sleeve 426 has a distal opening 440 to provide access to central bore 433. In addition, sleeve 426 has an aperture 435 on the exterior surface that provides access to central bore 433.

The proximal portion 431 of sleeve 426 includes a thumbwheel 444. Thumbwheel 444 provides a mechanism to translate the adapter 428 along longitudinal axis L relative to sleeve 426. Specifically, thumbwheel 444 may be rotated about longitudinal axis L such that rotation translates adapter 428 along longitudinal axis L towards the distal portion 436 of sleeve 426. As an alternative mechanism (not shown), a scroll wheel may be used that enables one to scroll the adapter 428 along longitudinal axis L towards the distal portion 436 of sleeve 426. Other mechanisms may be used to translate adapter 428 along longitudinal axis L relative to sleeve 426 as may be known to one skilled in the art.

Also, shown in FIGS. 17 and 18 is the adapter 428. The adapter 428 has an elongated body along longitudinal axis L. The elongated body is generally cylindrical in shape, but other cross-sectional shapes are considered, but not limited to triangular, square, hexagonal, elliptical, and tapered. Adapter 428 has a proximal portion 452 and a distal portion 454. Regardless of the cross-sectional shape of adapter 428, it is configured to be inserted into the central bore 433 of sleeve 426.

The distal portion 454 of adapter 428 has a housing 446 that is located within central bore 433 of sleeve 426. As will be discussed in more detail below, the housing 446 is designed to secure a bit portion 470 of material conduit 438 to adapter 428.

Also shown in FIGS. 17 and 18, is the material conduit 438. The material conduit 438 has a flexible elongated body 439. The elongated body 439 is generally cylindrical in shape, but other cross-sectional shapes are considered, but not limited to triangular, square, hexagonal, elliptical, and tapered. Furthermore, material conduit 438 has a proximal opening 462 and distal opening 466 that provides access to a central bore 460 that extends through the length of material conduit 438.

The material conduit 438 has a proximal portion 448 and a distal portion 450. The proximal portion 448 extends outward from the central bore 433 of the sleeve 426 through aperture 435. The proximal portion 448 includes a delivery system interface 458. The delivery system interface 458 allows a delivery system (not shown) to be attached to material conduit 438 to be able to access central bore 460. For example, the delivery system may include a syringe and/or bone filler device to be connected to material conduit 438 in order to access central bore 460. The delivery system interface 458 may be a luer connection, a threaded connection, or any other connection known in the art.

The distal portion 450 of material conduit 438 extends through aperture 435 of sleeve 426 into bore 433. The distal portion 450 of material conduit 438 includes the bit 470. Bit 470 has a proximal portion 429 and a distal portion 430. Specifically, the proximal portion 429 of bit 470 is secured by engagement mechanism 442 of housing 446 to adapter 428.

The distal portion 430 of bit 470 contains an elongated tubular shaped tip 432. Although bit 470 shown in FIG. 18 has a elongated tubular shaped tip 432, other configurations for the tip 432 may be utilized. Additionally, bit 470 has a central bore 434 extending along longitudinal axis L through the entire longitudinal length of bit 470. Furthermore, bit 470 has a proximal opening and a distal opening to allow access to central bore 434.

Additionally, bit 470 has projections 471 that are located near tip 432. Projections 471 extend from the exterior surface of bit 470 and taper toward longitudinal axis L.

In an alternative embodiment, bit 470 may be a separate component from material conduit 438. In such an embodiment, the distal portion 450 of the material conduit 438 includes a coupling element which provides a push-fit interface for releasably coupling the material conduit 438 with bit 470. Specifically, the coupling element is push-fit into central bore 434 of bit 470 to form a seal between material conduit 438 and bit 470. In other embodiments, the coupling element of material conduit 430 may be coupled with bit 470 by threaded connections, snap-fit, sonic welding, and any other method that one skilled in the art may utilize.

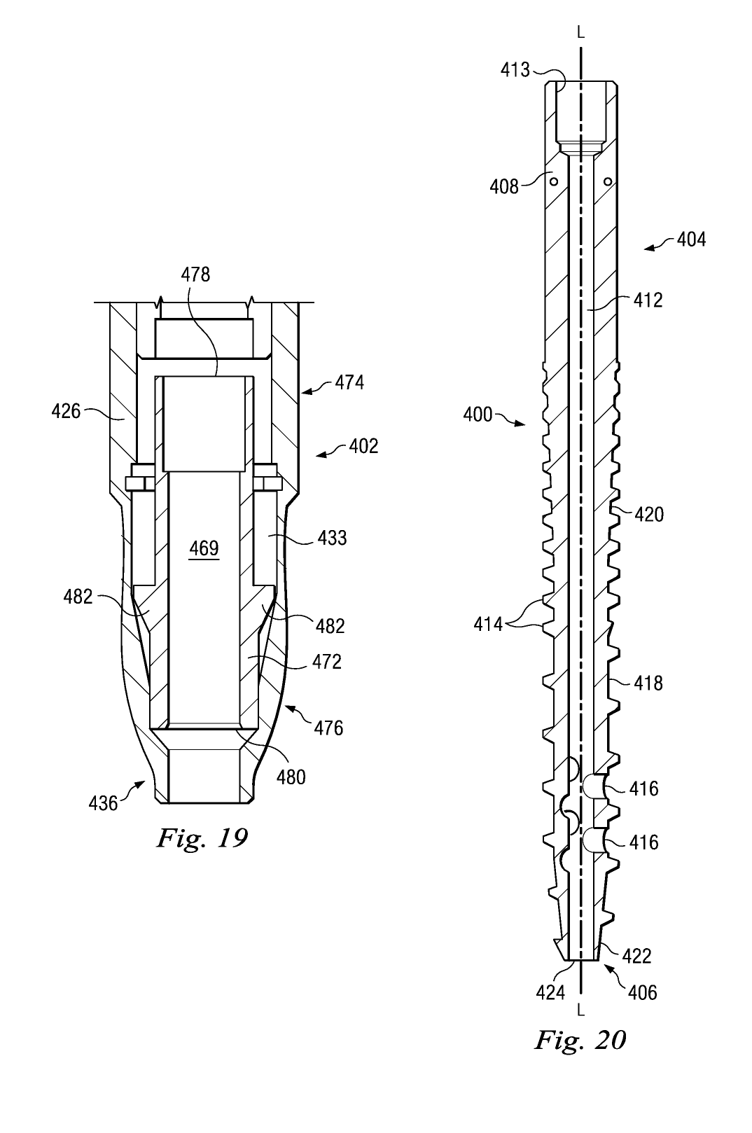

As shown within FIG. 19, coupling element 472 is housed within the central bore 433 in the distal portion 436 of sleeve 426. Coupling element 472 releasably couples the driver 402 to a screw (not shown). The coupling element 472 has a proximal portion 474 and a distal portion 476. The inner surface of the coupling element 472 forms the sidewalls of a central bore 469 extending therethrough along longitudinal axis L. The central bore 469 has a proximal opening 478 and a distal opening 480. Additionally, the central bore 469 is configured to receive bit 470 (not shown) through proximal opening 478. Finally, the exterior surface of the coupling element 472 has projections 482. Projections 482 contact the inner surface of sleeve 426 to prevent the distal portion 476 of the coupling element 472 from expanding away from longitudinal axis L.

FIG. 20 shows a cross-sectional view of an alternative bone fastener such as bone screw 400 which may be coupled with coupling element 472. Screw 400 has a an elongated body 420 along longitudinal axis L. The elongated body 420 has a proximal portion 404 and a distal portion 406. The proximal portion 404 includes a post 408. The post 408 in this exemplary embodiment is cylindrically shaped with an exterior surface that is smooth (i.e. non-abrasive). In other embodiments, the post 108 may be, but not limited to, flat, conical, balled and any other shape that may be considered by one having skill in the art. Additionally, the exterior surface may be, but not limited to, roughened, abrasive, indented, scalloped, threaded, and any other texture that may be considered by one having skill in the art

The post 408 has a proximal opening 413 to provide a passageway into a central bore 412. Central bore 412 extends longitudinally within screw 400 from the proximal portion 404 to the distal portion 406. In addition, proximal opening 413 is shaped to correspond to the shape of tip 432 of bit 470, such that a seal may be formed when tip 432 is inserted into central 412. In this exemplary embodiment, proximal opening 413 is circular shaped. Although other configurations for proximal opening 413 are considered and may be implemented by one having skill in the art to allow the tip 432 of bit 470 to engage proximal opening 413.

The elongated body 420 further comprises threads 414 to help secure the screw 400 within a bone. Near the distal portion 406 of screw 400, fenestrations 416 provide window like openings that form passageways between central bore 412 and the exterior surface 418 of screw 400. Although shown as four fenestrations within FIG. 20, fenestrations 416 are not limited to four and can be any number of fenestrations. Furthermore, fenestrations 416 may be located anywhere along the exterior surface 418 including on opposite sides of the elongated body 420. Finally, the fenestrations 416 shown in FIG. 20 are circular in shape, but other shapes are considered such as oval, square, and elliptical.

The distal portion 406 of screw 400 includes tip 422. The tip 422 has a distal opening 424 that provides access to central bore 412. As will be discussed in more detail below, central bore 412 allows substances to be injected into screw 400 using driver system 402. For example, once screw 400 has been inserted into a bone a filling composition such as cement can be injected through driver 402 into the central bore 412. Upon injection, the filling composition progresses though central bore 412 towards distal portion 406 and exits the bore at fenestrations 116 and the distal opening 124. Once the filling composition exits bore 412 it cures and bonds screw 400 to the bone. It should be noted that any number of filling compositions discussed above may be injected by driver 402 into screw 400.

All of the embodiments disclosed herein in whole or in part may be constructed of biocompatible materials of various types including metals or polymers. Examples of materials include, but are not limited to, non-cobalt-chromium alloys, titanium alloys, nickel titanium alloys, and/or stainless steel alloys, any member of the polyaryletherketone (PAEK) family such as polyetheretherketone (PEEK), carbon-reinforced PEEK, or polyetherketoneketone (PEKK); polysulfone; polyetherimide; polyimide; ultra-high molecular weight polyethylene (UHMWPE); and/or cross-linked UHMWPE. In one exemplary embodiment the adapter 428 may be formed all or in part of a metal and the sleeve 426 may be formed all or in part of a polymer.

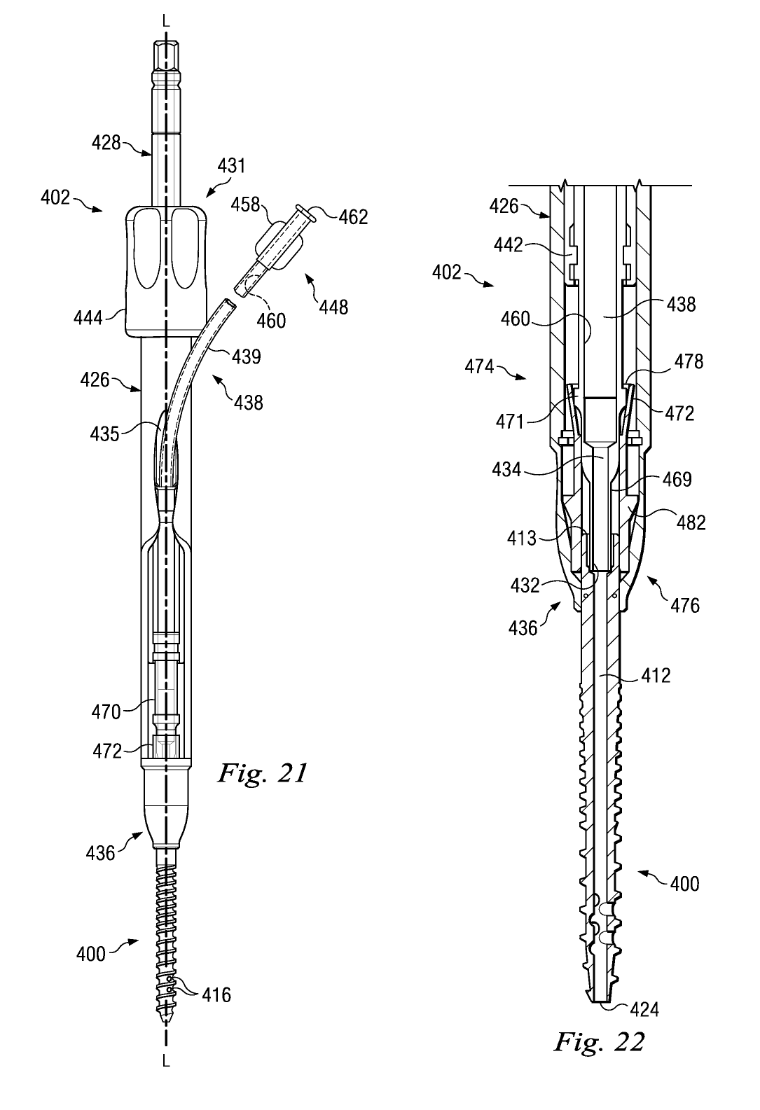

FIGS. 21 and 22 shows an exemplary embodiment of screw 400 engaged with driver 402. Screw 400 and driver 402 are similar to those of FIGS. 17-20 and identical structures and components are given the same reference numerals. As previously mentioned, driver 402 can be releasably coupled to screw 400 through coupling element 472.

As shown in FIGS. 21 and 22, thumbwheel 444 has been manipulated to translate adapter 428 along longitudinal axis L towards the distal portion 436 of sleeve 426. Because bit 470 is secured to adapter 428 via engagement mechanism 442, translation of adapter 428 also translates bit 470 along longitudinal axis L. As shown in FIG. 22, adapter 428 has been translated along longitudinal axis L such that bit 470 has been inserted into the proximal opening 478 of the coupling element 472 with the tip 432 extending through central bore 469 into the proximal opening 413 of the screw 400.

Insertion of bit 470 within central bore 469 causes the tapered projections 471 to slide along the inner surface of the proximal portion 474 of coupling element 472. The projections 471 cause the proximal portion 474 of coupling element 472 to expand away from longitudinal axis L. In turn, because projections 482 of the coupling element 472 prevent the distal portion 476 of the coupling element 472 from expanding away from longitudinal axis L, the distal portion 476 of the coupling element moves towards longitudinal axis L to couple screw 400 with driver 402.

The extension of tip 432 into the proximal opening 413 of screw 400 may form a seal between bit 470 and screw 400. Upon insertion of tip 432 into the proximal opening 413 of screw 400, the central bores 460, 434, and 412 of material conduit 438, bit 470, and screw 400 respectively are concentrically aligned to form a continuous bore extending from the proximal opening 462 of the material conduit 438 to the distal opening 424 of the screw 400. As previously mentioned, driver 402 can be used to inject substances, via material conduit 438, into screw 400. For example, a delivery system such as syringe 208 as discussed above, filled with flushing material may be attached to material conduit 438 at delivery system interface 458 to inject flushing material through central bores 460, 434, and 412. The injection of the flushing material removes any bone particles that may be blocking fenestrations 416 and/or distal opening 424 of the screw 400.

Additionally, a material delivery system such as bone filler device, as discussed above, may be attached or inserted into material conduit 438 through the proximal opening 462 of central bore 460. It should be noted that any number of the filling compositions discussed above may be injected by driver 402 into screw 400.

A bone filler device 210 attached to driver 402 is used to inject a filling composition such as cement through driver 402 via central bores 460 and 434 into central bore 412 of the screw 400. As previously discussed, fenestrations 416 and distal opening 424 of the screw 400 allow the filling composition to exit central bore 412. As the filling composition passes out of the central bore 412, the filling composition may engage the various pores, concavities and interstices of the bone structures surrounding screw 400, thereby creating a mass or collection of filling composition about the screw 400. After curing, the filling composition creates a firm fixation or anchoring of the screw 400 in a bone structure. Additionally, since the filling composition may tend to engage the various pores, concavities and interstices of a bone, the bone may tend to be strengthened by the infusion of filling composition through and around screw 400. Thus, driver 402 enables a system and method for securely anchoring screw 400 into bone to provide structural support and stabilization for damaged bones.

It should be noted that driver 402 and screw 400 may be decoupled from one another after injection. Specifically, thumbwheel 444 of sleeve 426 may be manipulated to translate adapter 428 along longitudinal axis L towards the proximal portion 431 of sleeve 426. Translation of adapter 428 along longitudinal axis L towards the proximal portion 431 of sleeve 426 causes bit 470 to disengage from central bore 469. Disengagement of bit 470 within central bore 469 causes the tapered projections 471 to slide along the inner surface of the proximal portion 474 of coupling element 472 away from screw 400 until they exit central bore 469. The exit of projections 471 from central bore 469 causes the proximal portion 474 of coupling element 472 to move back towards longitudinal axis L to a resting state. In turn, the distal portion 476 of the coupling element ceases from moving toward longitudinal axis L and returns to a resting state. Finally, when the proximal portion 474 and distal portion 476 of coupling element 472 are both in resting states then screw 400 and driver 402 are uncoupled from one another and driver 402 may be removed from screw 400.

In an alternative embodiment of a driver substantially similar to driver 402, the material conduit 438 may be omitted and the filling material may be passed through a cannular adapter which is connected to the bit. In such an alternative embodiment, the delivery system may be connected to an open end portion of the adapter to be able to access a central bore, which may be a material conduit, through the adapter. In this alternative embodiment, a thumbwheel may be manipulated to translate the adapter along longitudinal axis L. Because the bit is secured to the adapter, translation of adapter also translates the bit along longitudinal axis L until the bit extends into the proximal opening 413 of the screw 400. Upon insertion of bit tip into the proximal opening 413 of screw 400, the central bores of the adapter, the bit, and the screw respectively are concentrically aligned to form a continuous bore extending from the proximal opening of the adapter to the distal opening 424 of the screw 400. Thus, the alternative embodiment of driver enables a system and method for securely anchoring screw 400 into bone to provide structural support and stabilization for damaged bones.

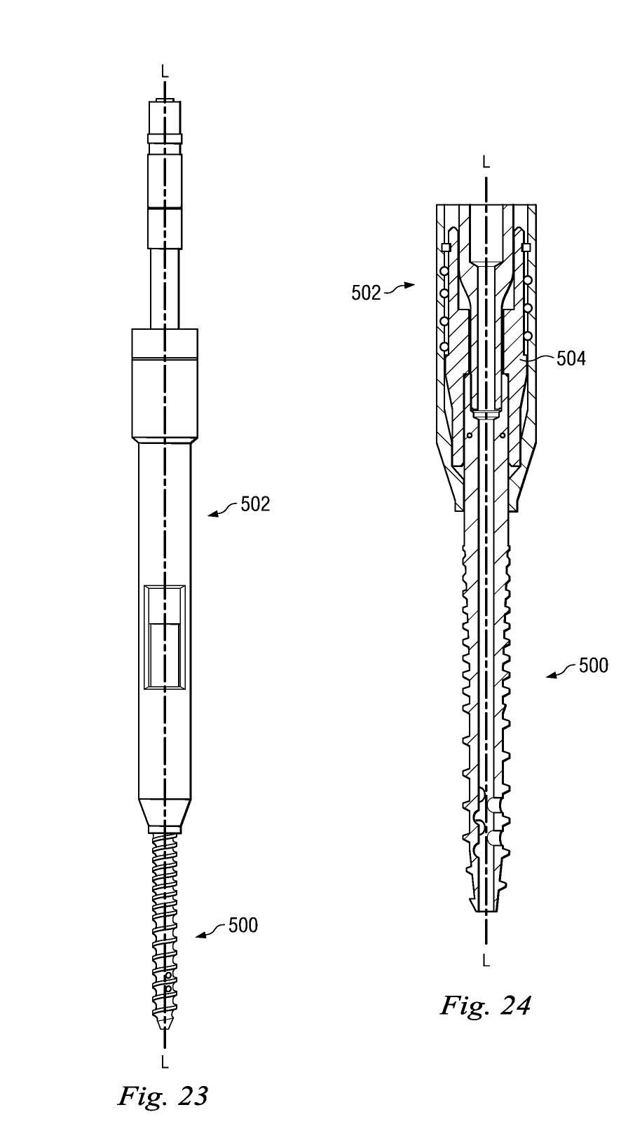

In yet another alternative embodiment, as shown in FIGS. 23 and 24, a driver labeled by reference numeral 502 is coupled with a screw labeled by reference numeral 500. Screw 500 is substantially similar to screw 400 seen in FIG. 20. Driver 502 is substantially similar to driver 125 seen in FIGS. 3-12 except for the mechanism used to couple driver 502 with screw 400. Driver 502 has an coupling element 504 that is substantially similar to coupling element 472 seen in FIG. 19. Coupling element 504 enables driver 502 to be releasably coupled with screw 500 in order to drive screw 500 into a bone structure and inject screw 500 with a filling composition. It should be noted that because of the rigidity of driver 502 a higher viscosity of filling composition may be injected through driver 502 into screw 500 than compared to legacy methods such as using a syringe to inject filling composition. Specifically, a material delivery system such as bone filler device 210 may be inserted into driver 502 in a similar manner as describe with respect to driver 125 in order to inject screw 500 with a higher viscosity filling composition.

While some embodiments of the present disclosure may be applied to the lumbar spinal region, embodiments may also be applied to the cervical or thoracic spine or within other bone structures. Other bone structures that the disclosed embodiments may be applied to include, but not limited to, a femur, tibia, fibula, humerus, radius, ulna, phalanges, clavicle, and any of the ribs.

While the present invention has been illustrated by the above description of embodiments, and while the embodiments have been described in some detail, it is not the intention of the applicant to restrict or in any way limit the scope of the invention to such detail. Additional advantages and modifications will readily appear to those skilled in the art. Therefore, the invention in its broader aspects is not limited to the specific details, representative apparatus and methods, and illustrative examples shown and described. Accordingly, departures may be made from such details without departing from the spirit or scope of the applicant's general or inventive concept. It is understood that all spatial references, such as “longitudinal axis,” “horizontal,” “vertical,” “top,” “upper,” “lower,” “bottom,” “left,” and “right,” are for illustrative purposes only and can be varied within the scope of the disclosure.

What is claimed is:

1. A driver for fastening a bone fastener to a bone, the driver comprising:
an elongated outer member including a first bore extending therethrough along a longitudinal axis and a coupling element that is releasably coupled with the bone fastener;
an elongated material conduit extending at least partially within the first bore, the material conduit including a second bore extending therethrough; and
a driving body with a driver head shaped to releasably engage the bone fastener, wherein the driving body includes a distal opening in communication with the second bore to allow the passage of a filling composition through the second bore and through the distal opening.

2. The driver of claim 1, further comprising an inner elongated member extending into the first bore, wherein the inner member further comprises a driving tool engagement interface shaped to engage a driving tool for rotating the driver about the longitudinal axis.

3. The driver of claim 2, wherein the inner member is linearly movable within the first bore along the longitudinal axis.

4. The driver of claim 1, wherein the driving body is formed from a first material extending at least partially through an elongated sleeve formed from a second material, the elongated driving body including the driver head, wherein the first material is more rigid than the second material

5. The driver of claim 2, wherein the outer member further comprises a thumbwheel engaged with the inner member for linearly moving the inner member along the longitudinal axis relative to the outer member.

6. The driver of claim 2, wherein the inner elongated member is the elongated material conduit.

7. The driver of claim 1, wherein the coupling element comprises a threaded portion sized to threadably engage the fastening member.

8. The driver of claim 1, wherein the coupling element comprises a tab sized to engage an indentation of the fastening member.

9. The driver of claim 1, wherein the coupling element comprises of a crimped portion sized to releasably engage the fastening member.

10. The driver of claim 1, wherein the driver head has a torx shape.

11. The driver of claim 1, wherein the material conduit further comprises a material delivery adapter sized to engage a material delivery device for dispensing the filling composition.

12. The driver of claim 1, wherein the driver head is push-fit into a distal portion of the inner member.

13. A system for stabilizing a bone, the system comprising:
a fastener including a head including a proximal opening, an elongated shaft, a first bore extending through the elongated shaft along a longitudinal axis, and an engagement member;
a driver comprising:
an outer member including a second bore extending therethrough along the longitudinal axis, the outer member including a coupling element releasably couplable with the engagement member of the fastener; and
a inner member, extending into the second bore and rotatable with respect to the outer member, including a third bore extending therethrough along the longitudinal axis and a driver head releasably couplable to the proximal opening of the fastening member,
wherein the coupling of the driver head with the proximal opening of the fastener concentrically aligns the first and third bores for passage of a filling composition therethrough.

14. The system of claim 13, wherein the elongated shaft of the fastener has an exterior surface including at least one thread and at least one fenestration.

15. The system of claim 13, wherein the inner member further comprises a material delivery adapter sized to engage a material delivery device for dispensing the filling composition.

16. The system of claim 13, wherein the inner member further comprises a driving tool engagement interface shaped to engage a driving tool for rotating the inner member about the longitudinal axis.

17. The system of claim 13, wherein the engagement member is pivotally attached to the head to allow multiaxial positioning of the fastener.

18. The system of claim 13, wherein the outer member further comprises a thumbwheel engaged with the inner member, wherein rotation of the thumbwheel linearly moves the inner member along the longitudinal axis relative to the outer member.

19. A method for securing a fastener into a bone, the method comprising the steps of:
coupling an elongated driving member to a bone fastener along a longitudinal axis, wherein the bone fastener includes a first bore in communication with at least one fenestration and the driving member includes a second bore and further wherein coupling the driving member and the bone fastener concentrically aligns the first and second bores about the longitudinal axis;
rotating the bone fastener about the longitudinal axis to threadably engage the fastener with the adjacent bone; and
delivering a bone filling composition into the second bore of the driving member for passage through the second bore, the first bore of the fastener, and out the at least one fenestration.

20. The method of claim 19, further comprising flushing the concentrically aligned first and second bores with saline to alleviate blockage within the first and second bores.

21. The method of clam 19, wherein the bone filling composition is a bone cement.

22. The method of claim 19, wherein the driving member comprises an inner elongated member and an outer elongated member and the step of coupling further includes connecting the outer elongated member to a pivotable engagement portion of the bone fastener and connecting the inner elongated member to the bone fastener and the step of rotating includes rotating the inner elongated member and the bone fastener with respect to the outer elongated member and with respect to the pivotable engagement portion.

23. The method of claim 22 further including linearly moving the inner elongated member with respect to the outer elongated member to connect the inner elongated member to the bone fastener.

24. The method of claim 19, further comprising retracting the driver head of the driving member along the longitudinal axis way from the first bore to decouple to the engagement member from the engagement surface.

25. The method of claim 19, further comprising manipulating a thumbwheel of the driving member to lower the driver head into the first bore of the bone fastener.
